(19) United States
(12) Patent Application Publication
(10) Pub. No.: US 2019/0038844 A1
(43) Pub. Date: Feb. 7, 2019

(54) Method for Checking the Condition of a Therapeutic Agent Housed in an Injection Device

(71) Applicant: EMPERRA GMBH E-HEALTH TECHNOLOGIES, Potsdam (DE)

(72) Inventors: Janko SCHILDT, Potsdam (DE); Markus BENTRUP, Berlin (DE)

(21) Appl. No.: 16/081,735

(22) PCT Filed: Feb. 8, 2017
(86) PCT No.: PCT/EP2017/052707

§ 371 Date: Aug. 31, 2018

(30) Foreign Application Priority Data
Mar. 7, 2016 (DE) 10 2016 104 101.7

(51) Int. Cl. A61M 5/315; G01P 15/14

(52) U.S. Cl. CPC A61M 5/31568; A61M 5/3157; A61M 2005/3125; A61M 2205/332; G01P 15/14

(57) ABSTRACT

The invention relates to an injection device for injecting dosed amounts of a liquid therapeutic agent, comprising a receiving system for the therapeutic agent, an application system for transferring the therapeutic agent to an application site, a dosing system for transferring the therapeutic agent from the receiving system to the application system, a trigger system for activating the dosing system, and a detection system for detecting the amount of the therapeutic agent applied. According to the invention, a sensor arrangement comprises at least one recognition system which detects, prior to injection, whether the therapeutic agent has been mixed.

### CROSS-REFERENCE TO RELATED APPLICATIONS

This application is a Nationalization of PCT Patent Application Serial No. PCT/EP2017/052707 filed on Feb. 8, 2018, entitled “Method for Checking the Condition of a Therapeutic Agent Housed in an Injection Device,” which application is expressly incorporated herein by reference in its entirety.

### BACKGROUND

The invention relates to a method for checking the condition of a therapeutic agent housed in an injection device.

Many medications (therapeutic agents) need to be injected into the body. This applies above all to those medications that, when administered orally, are inactivated or lose their effectiveness to a decisive degree. These medications include, in particular, proteins, such as insulin, carbohydrates, such as heparin, antibodies, or most vaccines. For injection into the body, syringes, medication pens or medication pumps are usually used.

With insulin therapy, in particular with intensified, conventional insulin therapy, and with conventional insulin therapy, insulin is not applied in constant quantities. In the case of conventional insulin therapy, insulin is applied at certain times of the day. The daily routine of the patient is oriented to these times. In the case of intensified, conventional insulin therapy, a basic insulin requirement is provided, often through insulin, known as basal insulin, that acts slowly and over a long period of time.

At mealtimes, fast-acting insulin is injected. The dose of the fast-acting insulin is essentially oriented to the carbohydrates that have been eaten. The dose is therefore specifically selected depending on external circumstances. Such circumstances include the time of day, the amount of exercise taken, nutrition, and the like.

Diabetes mellitus can have severe long-term consequences and cause harm to the body. This can be significantly reduced by means of adapted insulin therapy, preferably an intensified, conventional insulin therapy. However, an incorrect dose can also lead to short-term consequences such as hypoglycaemia. The best possible adjustment of the dose to the respective circumstances is therefore particularly desirable.

For this reason, diabetes mellitus patients are required to precisely log their daily habits and the doses of insulin they have administered. Such a log usually comprises the measured blood sugar level, the quantity of carbohydrates eaten, the injected dose of insulin, and the date and time. The doctor treating the patient or the patient themselves can refer to the log to determine or adjust the respective dose. Thus, high dosing precision is particularly important for insulin.

A widely used injection device for injecting dosed quantities of insulin is the so-called insulin pen. In contrast to insulin syringes, an exchangeable medication container is used in the case of the insulin pen. This container, also known as a carpule or ampule, is delivered filled with insulin by the manufacturer and is inserted into the insulin pen before use. When the pen is first used, a needle pierces the sealing disc of the ampule and, when the insulin is applied, administers the parenteral injection of the pre-selected dose. During the injection, an injection and triggering mechanism generates an injection stroke, which causes the forward feed of a piston or plug in the ampule and leads to the discharge of the pre-selected dose into the target tissue. The mechanism usually consists of a piston rod with a construction length that corresponds to the ampule plug stroke.

Known insulin pens have a similar external appearance to a thicker ball-point pen. They comprise a housing in which the ampule containing the insulin can be held. The ampule is usually replaceable. However, arrangements are also known that are designed as disposable pens. The ampules and their content, dimensions and handling are not standardised. An ampule from one manufacturer cannot therefore usually be inserted into the pen of another manufacturer.

An insulin pen is known from EP 2 414 009 B1 that enables an adapter arrangement for adaptation to ampules of different dimensions and content.

A pen comprises a dosage system. The dose required is set on a dosage button. This is then injected into the subcutaneous fatty tissue by means of an injection system, which can be designed with or without a needle. Insulin pens are known in which the set dose is displayed on a display instead of on a mechanical display on the dosage button. The display is supplied with energy by means of a voltage source integrated in the insulin pen. The patient can set the dose and note this in their diabetics' journal.

Insulin pens are known in which the logging is automatically conducted in a detection system integrated into the insulin pen. This is connectable with a data processing unit via a data connection that can be wired or wireless. With regard to the structure and function of such an insulin pen, reference is made to the disclosure of WO 2013/079644 A1.

Insulin pens are divided into single-use “disposable” and multiple-use “reusable” insulin pens. In the case of disposable insulin pens, the ampule and the dosage mechanism form a unit that is prefabricated by the manufacturer and are disposed of together after the ampule has been emptied. No provision is made for a re-use of the dosage mechanism. Reusable insulin pens present the user with greater challenges. When the ampule is replaced, the piston rod must be returned to the initial position. Depending on the model, this occurs by turning or pushing the piston rod while at the same time activating a special function in the dosage mechanism.

Reusable insulin pens are further divided into manual and semi-automatic insulin pens. In the case of manual insulin pens, the user uses their finger pressure to actuate an injection button, thus determining the duration and progress of the injection. In the case of semi-automatic insulin pens, by contrast, a spring is manually tensioned before use, which stores the necessary injection energy. The spring is unlocked by the user during the actual injection procedure.

The therapeutic agent (insulin) present in the ampule is usually used for a plurality of injections. This means that for each injection, only a partial quantity of the therapeutic agent present in the ampule is injected. Time periods of different lengths lie between the individual injections. As a result, the therapeutic agent present in the ampules de-mixes due to the different densities of the active substances or filling substances contained in them. It is therefore necessary to thoroughly mix the therapeutic agent before each injection in order to provide an adequate effect. If this mixing does not occur, erroneous effects of the injected therapeutic agent may result.

US 2016/0030683 A1 discloses an injection system for injecting dosed quantities of a fluid therapeutic agent which comprises a sensor element. By means of this sensor element, different parameters of the therapeutic agent or of the system itself can be detected and added to an evaluation.

US 2005/043676 A1 discloses an injection system for injecting dosed quantities of a fluid therapeutic agent which comprises an acceleration sensor. This acceleration sensor is used to detect incorrect handling of the injection system, as a result of which the injection system is subjected to major knocks. As a result, for example, a falling down of the injection system which leads to internal damage to the device itself or to an ampule receiving the therapeutic agent, or the like, can be detected,. When an acceleration limit value is exceeded, a non-reversible display is triggered, which clearly shows a patient that the injection device has been subjected to an impermissible mechanical load.

### BRIEF SUMMARY

The object of the invention is to create a method of the generic type, with which it can be detected in a simple manner whether a sufficient mixing of the therapeutic agent has occurred prior to an injection.

According to the invention, the object is attained by means of a method with the features described in claim 1. Due to the fact that a movement of a housing of the injection device is measured, the signals that correspond to the measured movement are transferred to a detection system for detecting states of the injection device, the detection system evaluates a measured acceleration over time and generates a signal when a specifiable limit value is exceeded, it is advantageously possible to determine a mixing of the therapeutic agent. In other words, a mixing of the therapeutic agent is required which generates a corresponding signal. As a result, the patient is rendered able to detect whether they have achieved a correct mixing of the therapeutic agent prior to injection. As a result, the necessary administration of therapeutic agent is overall ensured, or at least increased.

In a further preferred embodiment of the invention, it is provided that an acceleration curve of a movement of the injection device is recorded as a degree of mixing of the therapeutic agent, and is taken into account when the administration of the therapeutic agent is logged. As a result, it is advantageously possible that the corresponding information is available to both the patient and a doctor evaluating the correct administration of the therapeutic agent. As a result, a more optimum dosage of the therapeutic agent is possible overall.

Further preferred embodiments of the invention emerge from the other features named in the sub-claims.

### BRIEF DESCRIPTION OF THE DRAWINGS

The invention will now be explained in greater detail below in an exemplary embodiment, with reference to the related drawings, in which:

FIG. 1 shows a schematic view of an injection device, and

FIG. 2 shows an exemplary signal curve

### DETAILED DESCRIPTION

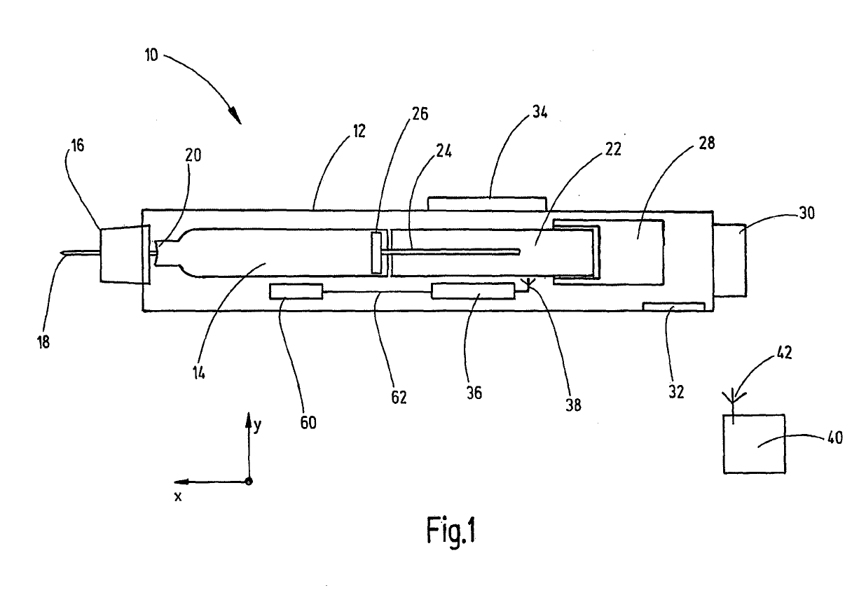

FIG. 1 shows a schematic view of an injection device designated overall with the numeral 10. The structure and function of injection devices 10 are generally known, so this is not described in greater detail within the scope of the present description. This can for example be an injection device with or without a needle.

The injection device can also be a disposable or reusable injection device. Furthermore, the injection device can be equipped with or without an adapter for receiving different ampules from different manufacturers.

The injection device 10 has a housing 12, within which a receiving system 14 is arranged for receiving a therapeutic agent to be injected, with insulin being assumed below. The receiving system 14 can be an ampule or a carpule.

The injection device 10 further comprises an application system 16 for transferring the insulin to an injection site (application site). The injection site is for example an area of skin of a patient. The application system 16 can have a pin needle 18 for this purpose, which is pierced into the skin of the patient. The application system 16 is arranged on the housing such that it is replaceable, and punctures a membrane 20 of the receiving system 14 with the pin 18.

The injection device 10 further comprises a dosing system 22, which has an actuation element 24 that is in active contact with a plug 26 of the receiving system 14.

A trigger system 28 is assigned to the dosing system 22. The trigger system 28 interacts with the dosing system 22.

The injection device 10 further comprises a dosage button 30, via which the dose of insulin to be injected can be set. Further, a trigger button 32 is provided, which is operatively connected to the trigger system 28.

The injection device 10 further comprises a display 34, which is equipped with a display field.

The injection device 10 further comprises a detection system 36, which is connectable via an interface 38 with a data processing unit 40 that is only briefly mentioned here. The data processing unit 40 also has an interface 42, which can communicate with the interface 38. The connection can here be wired or wireless.

The injection device 10 further comprises a movement sensor 60. The movement sensor 60 is for example a so-called gyrosensor. This has a sensitive system, such as a correspondingly positioned seismic mass, with which a detection is made as to whether the movement sensor 60 and thus the injection device 10 is moved in at least one spatial direction. Here, via the movement sensor 60, an acceleration can in particular be recorded in the three spatial directions, which also results from a turning, shaking, moving to and fro, and the like of the injection device. The three spatial directions refer on the one hand to the x direction, which here coincides with the longitudinal extension of the injection device 10. On the other hand, this is the y direction, which coincides with the height extension of the injection device, and further the z direction, which coincides with the depth of the injection device 10 (in the relevant depiction in FIG. 1).

The structure and operating principle of such gyrosensors are generally known, so these are not discussed in greater detail within the scope of the present invention. Of decisive importance is the fact that the corresponding gyrosensor supplies the acceleration signals in accordance with the actual acceleration directions and the acceleration height of the injection device 10.

The movement sensor 60 is connected with the detecting system 36 via a connecting line 62. The movement sensor 60 and the connecting line 62 can be integrated into the housing 12 of the injection device 10, so that these have a defined position in relation to the housing 12.

The injection device 10 shown in FIG. 1 shows the following function:

When the injection device 10 is used as determined, before the actual injection, a mixing of the therapeutic agent (insulin) should be conducted. This mixing serves to evenly distribute the active substances in the receiving system 14, so that with a subsequent injection, a defined active substance administration can occur.

For an intended injection, the quantity of insulin to be dosed is set using the dosage button 30. The set quantity can be read on the display 34 and can thus be monitored. After the injection device 10 has been placed onto the skin of the patient to be treated with the pin 18 of the application system 16, the trigger button 32 is actuated. Then, by means of the trigger system 28, the injection procedure is triggered, whereby the actuating element 24 of the dosing system 22 is charged with a propulsion force. As a result, the actuating element 24 displaces the plug 26 within the receiving system 14, so that the desired set dose of insulin can be injected via the application system 16. Such a structure and such a function of the injection device 10 are known in principle.

By means of the movement sensor 60, therefore, the accelerations are detected in at least one spatial direction, preferably in all three spatial directions, of the injection device 10. The corresponding signals are fed to the detection system 36 via the connecting line 62. The detection system compares the signals delivered by the movement sensor 60 with empirical values stored in a storage unit, and from this, determines the degree of mixing that has occurred of the therapeutic agent (insulin) present in the receiving system 14. Here, an evaluation of the delivered signals can be conducted both according to level (amplitude) and over time. Here, different evaluation criteria are stored. In general, it can be said that the higher the amplitude, the higher the acceleration, and the lower the time required in order to achieve the desired degree of mixing.

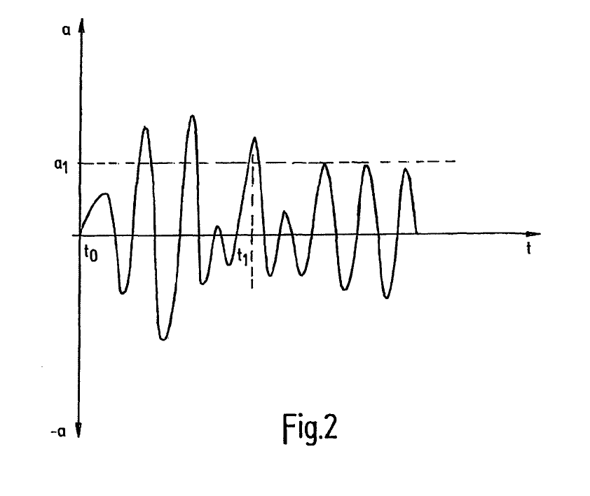

In FIG. 2, a measurement signal delivered by the movement sensor 60 is shown as an example. Here, the acceleration a is spread over time t. For example, here, the purpose is to show acceleration a in the x direction, i.e. in the longitudinal extension of the injection device 10. By moving the injection device 10 back and forth, accelerations in the x direction occur, as well as in the direction opposite to the x direction, i.e. positive and negative acceleration. These fluctuate around the zero line. In accordance with the strength of the movement, very different acceleration values emerge over time. By means of the detection system 36, the measured signal is compared with at least one required signal value. For example, here, an anticipated signal value is entered for the acceleration al. If it is now determined that this limit value al has been exceeded multiple times within a specific time frame, such as t0 to t1, a sufficient mixing of the therapeutic agent can be determined. The detection system 36 will then generate a corresponding signal to the display 34 and then, sufficient mixing is shown visually by means of an appropriate symbol. An acoustic signal can also be given, either in addition or exclusively.

Further, the detection system 36 can be designed in such a manner that an injection of the injection device 10 is only released when sufficient mixing is detected.

The corresponding signals can also be transferred to the data processing unit 40 for later evaluation by the patient and/or a doctor. In this way, it can be determined whether sufficient mixing really has occurred in the case of the individual injections. From this, a conclusion can be drawn regarding the actual active substance injection on the patient, and taken into account for the subsequent therapy.

Overall, it is achieved with the method according to the invention that the dosage precision is increased. Movements of the injection device 10 are reliably automatically detected as such and logged accordingly via the detection device 36. During the later evaluation of the logs by a treating doctor, a more precise adjustment can thus be made with reference to the quantity of insulin that has been applied in reality.

1. A method for checking the condition of a therapeutic agent housed in an injection device, characterized in that
a movement of a housing of the injection device is measured, the signals that correspond to the measured movement are transferred to a detection system for detecting states of the injection device, the detection system evaluates a measured acceleration over time and generates a signal when a specifiable limit value is exceeded.

2. The method according to claim 1, characterized in that
when the limit value is exceeded for a specified period of time, the signal is generated.

3. The method according to claim 1, characterized in that
when the limit value is exceeded at least twice within a specifiable period of time, the signal is generated.

4. The method according to claim 3, characterized in that
an acceleration curve is recorded and is taken into account when administration of the therapeutic agent is logged.

5. A method for checking the condition of a therapeutic agent housed in an injection device, the method comprising the acts of:
measuring movement of the injection device, generating movement signals that correspond to the measured movement, and transferring the movement signals to a detection system for detecting states of the injection device;
evaluating, by the detection system, a measured acceleration of the injection device over time; and
generating an output signal when the measured acceleration of the injection device equals or exceeds a predetermined value over a predetermined period of time.

6. The method according to claim 5, wherein the output signal is generated when the predetermined value is exceeded once during the predetermined period of time.

7. The method according to claim 5, wherein the output signal is generated when the predetermined value is exceeded twice during the predetermined period of time.

8. The method according to claim 6 or claim 7, wherein an acceleration curve is recorded and is taken into account when administration of the therapeutic agent is logged.
